(19) United States
(12) Patent Application Publication
(10) Pub. No.: US 2026/0296996 A1
(43) Pub. Date: Oct. 1, 2026

(54) METHODS FOR PRODUCING 1,1-DIFLUOROPROPENE (HFO-1252ZC) FROM 1,1,1-TRICHLOROPROPANE (HCC-260FB)

(71) Applicant: Solstice Advanced Materials US, Inc., Morris Plains, NJ (US)

(72) Inventors: Haiyou Wang, Amherst, NY (US); Selma Bektesevic, Williamsville, NY (US); Joshua DeMuth, Charlotte, NC (US); Vinayak Krishnamurti, East Amherst, NY (US); Chen Liang, Charlotte, NC (US); Akbar Mahdavi-Shakib, Buffalo, NY (US); Dimitrios Papanastasiou, Buffalo, NY (US)

(21) Appl. No.: 19/578,378

(22) Filed: Mar. 25, 2026

Related U.S. Application Data
(60) Provisional application No. 63/780,034, filed on Mar. 28, 2025.

(51) Int. Cl. C07C 17/25; C07C 17/35; C07C 17/383

(52) U.S. Cl. CPC C07C 17/25; C07C 17/35; C07C 17/383; C07C 2527/132; C07C 2527/133; C07C 2527/138; C07C 2527/14; C07C 2527/24

(57) ABSTRACT

A method for producing 1,1-difluoropropene (HFO-1252zc) from 1,1,1-trichloropropane (HCC-260fb) in the form of a two-step reaction is described. Step one of the reaction includes hydrofluorination of HCC-260fb to produce 1-chloro-1,1-difluoropropane (HCFC-262fc). Step two of the reaction includes dehydrochlorination of HCFC-262fc to produce HFO-1252zc. Step one includes a catalyst, and step two may include a catalyst.

### CROSS-REFERENCE TO RELATED APPLICATION

This application claims priority to U.S. Provisional Application No. 63/780,034 filed Mar. 28, 2025, which is herein incorporated by reference in its entirety.

### FIELD

The present disclosure relates generally to a method for producing 1,1-difluoropropene (HFO-1252zc), and more specifically to methods for producing HFO-1252zc from 1,1,1-trichloropropane (HCC-260fb).

### BACKGROUND

Fluorocarbon fluids have properties that are desirable for use as heat transfer media, immersion coolants, liquid or gaseous dielectrics, industrial refrigerants, and other applications.

For example, 1,1-difluoropropene (HFO-1252zc) has recently found increased utility for a variety of uses. Potential end use applications of HFO-1252zc include refrigerants, either used alone or in blends with other components, solvents for organic materials, heat transfer fluid, blowing agent, and as a chemical intermediate in the synthesis of other halogenated hydrocarbon solvents.

This is a need for developing new and useful methods for synthesizing HFO-1252zc.

### SUMMARY

The present disclosure provides a method for producing 1,1-difluoropropene (HFO-1252zc) from 1,1,1-trichloropropane (HCC-260fb) in the form of a two-step reaction. Step one of the reaction comprises hydrofluorination of HCC-260fb to produce 1-chloro-1,1-difluoropropane (HCFC-262fc). Step two of the reaction comprises dehydrochlorination of HCFC-262fc to produce HFO-1252zc. Step one comprises a catalyst, and step two may comprise a catalyst.

In one form thereof, the present disclosure provides a method for producing HFO-1252zc by reacting 1,1,1-trichloropropane (HCC-260fb) with hydrogen fluoride in the presence of a catalyst to produce a first product composition comprising 1-chloro-1,1-difluoropropane (HCFC-262fc).

In one form thereof, the second reacting step is conducted in vapor phase at a second temperature without the presence of a catalyst. In another form thereof, the second reacting step comprises reacting HCFC-262fc from the first product composition in the presence of a caustic solution to produce the second product composition.

### BRIEF DESCRIPTION OF THE DRAWINGS

FIG. 1 is a schematic process flow diagram for the conversion of 1,1,1-trichloropropane (HCC-260fb) to 1,1-difluoropropene (HFO-1252zc).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, “HFO-1252zc” refers to 1,1-difluoropropene.

As used herein, “HCC-260fb” refers to 1,1,1-trichloropropane.

As used herein, “HCFC-261fc” refers to 1,1-dichloro-1-fluoropropane.

As used herein, “HCFC-262fc” refers to 1-chloro-1,1-difluoropropane.

As used herein, “HFC-263fb” refers to 1,1,1-trifluoropropane.

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation when substantially pure reactant is used:

% conversion of molecule X=(100−molecule X mol. % in the organic components of a product mixture)

When the reactant includes impurities or recycled components, i.e., is a component in a reaction mixture, the conversion of a reactant molecule (molecule X) during a reaction is calculated using the following equations:

% conversion of molecule X=(change in X mol. %)/(X mol % in the reactant mixture)

OR

% conversion of molecule X=(X mol % in the reactant mixture−X mol % in the product mixture)/(X mol % in the reactant mixture)

As used herein, selectivity to a molecule formed during a reaction (molecule X) is calculated using the following equation:

% selectivity to molecule X=mol. % of molecule X in the organic components of a product mixture/(100−mol. % of reactant molecules in the organic components of a product mixture)×100.

### II. Overview

The present disclosure provides a method for producing 1,1-difluoropropene (HFO-1252zc) from 1,1,1-trichloropropane (HCC-260fb) according to a two-step process shown below (“Process 1”), which includes the following two steps: (i) hydrofluorinating HCC-260fb to produce 1-chloro-1,1-difluoropropane (HCFC-262fc), and (ii) dehydrochlorinating HCFC-262fc to produce HFO-1252zc.

Schematic equations for the two steps (“Step (i)”, “Step (ii) Vapor Phase”, and “Step (ii) Liquid Phase”) of Process 1 are represented below:

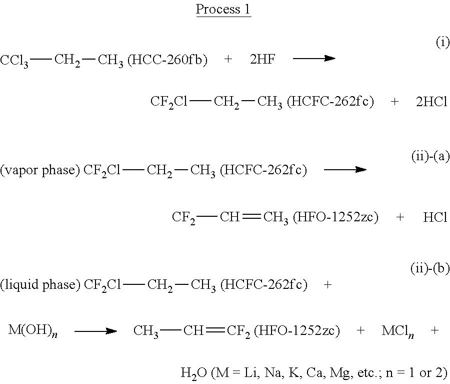

Both Step (i) and Step (ii) of Process 1 may proceed through either a vapor phase reaction or a liquid phase reaction. Step (ii)-(a) of Process 1 may be conducted in vapor phase via a pyrolysis process in a tubular reactor or an electric heater reactor. Step (ii)-(b) of Process 1 is conducted in vapor phase in a liquid phase reactor using a caustic solution.

Step (i) of Process 1 produces HCFC-262fc as a main product, as well as recyclable intermediates (e.g., CFCl₂—CH₂—CH₃ (HCFC-261fc)) and/or undesired byproducts (e.g., CF₃—CH₂—CH₃ (HFC-263fb)). Step (ii)-(a) and Step (ii)-(b) of Process 1 produces HFO-1252zc as a main product, with potential byproducts including, for example, CFCl═CH—CH₃ (HCFO-1251 or 1-chloro-1-fluoropropene), CF≡C—CH₃ (1-fluoropropyne or 1-fluoro-1-propyne), and CCl≡C—CH₃ (1-chloropropyne).

Further details regarding each of Steps (i), (ii)-(a), and (ii)-(b) are set forth below.

### III. Process Flow

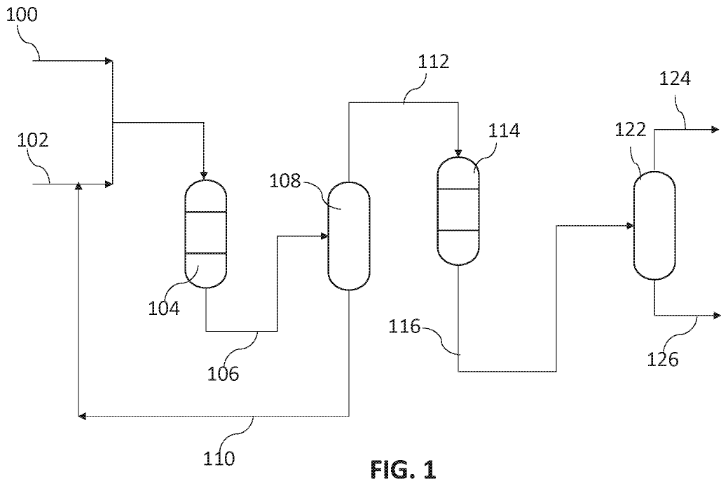

FIG. 1 is a schematic process flow diagram for the conversion of 1,1,1-trichloropropane (HCC-260fb) to 1,1-difluoropropene (HFO-1252zc).

Referring to FIG. 1, stream 100 comprising hydrogen fluoride (HF) and stream 102 comprising HCC-260fb enters reactor 104. Inside reactor 104, HCC-260fb undergoes hydrofluorination reaction of Step (i) to produce a first product composition comprising 1-chloro-1,1-difluoropropane (HCFC-262fc). Stream 106 including first product composition enters a separation unit 108 (which may comprise one or more distillation columns, scrubbers, adsorbers, absorbers, membrane separation, flash vessels, vaporizers, etc.) for producing stream 112 including predominantly HCFC-262fc and recycle stream 110.

The separation unit 108 may include, for a liquid phase reactor, a catalyst stripper connected to the reactor 104 for returning almost all of the entrained catalyst, and at least a portion of unconverted HCC-260fb and underfluorinated intermediates such as HCFC-261fc to reactor 104 for further hydrofluorination reaction. The separation unit 108 may also include an HCl column for separating and recovering HCl, an HF recovery unit for separating and recovering HF, a low pressure caustic solution tower for acid removal, concentrated sulfuric acid/molsiv drying column(s) for water removal, and distillation column(s) for isolating HCFC-262fc from the product stream 106 before entering second reactor 114 for Step (ii) reaction. Recycle stream 110 comprising unconverted HCC-260fb and HCFC-261fc may be directly recycled back to reactor 104.

Top stream 112 from separation unit 108 comprising HCFC-262fc enters second reactor 114 where HCFC-262fc undergoes Step (ii) dehydrochlorination reaction to produce HFO-1252zc. Optionally, top stream 112 may be sent to another distillation column to remove light components such as HFC-263fb from the top stream; the bottom stream comprising HCFC-262fc may be either sent to reactor 114, or collected in a storage tank. The second reactor 114 may optionally include a HCFC-262fc feed system for feeding the HCFC-262c from the storage tank to the second reactor 114.

The effluent stream 116 from second reactor 114 comprising HFO-1252zc enters second separation unit 122 for the separation of desired product HFO-1252zc from unreacted HCFC-262fc. The second separation unit 122 may include an HCl column for separating and recovering HCl, a low pressure caustic solution tower for acid removal, a concentrated sulfuric acid/molsiv drying column(s) for water removal, an HCFC-262fc recycle column to separate unconverted HCFC-262fc for recycling, and 1252zc purification columns (lights and product columns) to make high purity HFO-1252zc product.

Top stream 124 from second separation unit 122 contains about 99.9% of desired product HFC-1252zc. Bottom stream 126 from second separation unit 122 containing unreacted HCFC-262fc is collected or sent back to second reactor 114. Occasional/optional venting may be performed on stream 126 to prevent undesired byproduct accumulation in the system.

The hydrofluorination reaction of Step (i) of Process 1 is carried out in either liquid phase or vapor phase in a suitable reactor, for example a pressure vessel or a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The reactor 104 may be first cleaned and flushed with an inert gas such as nitrogen, followed by charging a catalyst such as those described below. The catalyst may be pretreated within the reactor such as by drying in the manner described further below, followed by metering the reactants into the reactor to initiate the reaction.

The dehydrochlorination reaction of Step (ii)-(a) may be carried out in vapor phase in a conventional bundle-type tubular reactor. Alternatively, the dehydrochlorination reaction of Step (ii)-(a) may be carried out in an electric heater reactor. The electric heater reactor includes a tube (e.g., a sheath), a metal alloy wire such as Nichrome wire, and a compacted metal oxide material (e.g., MgO) to heat the reactor tube to a desired outer surface temperature effective for the reaction to take place. The electric heating element may be packed with MgO powder to conduct heat from Nichrome wire while also acting as an electrical insulator. The manufacturing process includes inserting Nichrome wire into a tube (e.g., a sheath), packing with MgO powder, and rolling the tube again to compact the MgO and form a ceramic-like material. The MgO powder is isolated from the process because of the sheath tube wall throughout the interior of the heater. The sheath tubes may be constructed from a material which is resistant to temperature and/or corrosive effects of HF or HCl.

The dehydrochlorination reaction of Step (ii)-(b) may be carried out in liquid phase in a suitable liquid phase reactor, for example a reactor (e.g., a Parr® reactor) made from a material which is resistant to temperature, pressure and/or corrosion such as stainless steel, nickel and its alloys, including Hastelloy (for example, Hastelloy C276), Inconel (for example Inconel 600), Incoloy, and Monel wherein the vessels which may be lined with fluoropolymers.

The liquid-phase reactor may be first cleaned and flushed with deionized water, followed by charging with a basic solution made from metal hydroxides M(OH)ₙ (n=1 or 2), for example, alkali metal hydroxides (e.g., LiOH, NaOH, KOH) or alkaline earth metal hydroxides (e.g., Mg(OH)₂, Ca(OH), Sr(OH)₂), or mixtures thereof, and then adding organic feed to carry out the reaction. In another example, the reactor may be first cleaned, flushed with nitrogen, followed by charging with organic feed, and then adding a basic solution made from metal hydroxides M(OH)ₙ (n=1 or 2), for example, alkali metal hydroxides (e.g., LiOH, NaOH, KOH) or alkaline earth metal hydroxides (e.g., Mg(OH)₂, Ca(OH), Sr(OH)₂), or mixtures thereof.

The reaction conditions for each of Step (i) and Step (ii) in Process 1 are discussed in more details below.

### IV. Step (i)

As discussed above, Step (i) of Process 1 includes hydrofluorinating 1,1,1-trichloropropane (HCC-260fb) to produce 1-chloro-1,1-difluoropropane (HCFC-262fc). The feed (e.g., reactant composition) for Step (i) includes HCC-260fb. The catalyst and process conditions used in Step (i) hydrofluorination reaction of Process 1 is detailed below.

### Step (i) Catalyst

The Step (i) hydrofluorination reaction may be conducted in liquid phase the presence of a fluorination catalyst. A non-exhaustive list of such fluorination catalysts include, for example, Lewis acids, transition metal halides, Group IVb metal halides, Group Vb metal halides, or combinations thereof. Examples of liquid phase fluorination catalysts include antimony halide (e.g., SbCl₅, SbF₃,), a tin halide (e.g., SnCl₄), a tantalum halide, a titanium halide (e.g., TaCl₅), a niobium halide, or combinations thereof. The deactivated catalyst can be regenerated by using an oxidative agent such as Cl₂.

The Step (i) hydrofluorination reaction may be conducted in vapor phase in the presence of a solid fluorination catalyst. Such fluorination catalyst may include metal fluorides and fluorinated metal oxides. A non-exhaustive list of such fluorination catalysts include CrF₃, AlF₃, fluorinated metal oxides including Cr₂O₃, Al₂O₃, ZnO—Cr₂O₃ and their various mixtures. The deactivated catalyst may be regenerated by burning off the coke that is coating the surface of the catalyst.

### Step (i) Catalyst BET Surface Area

The catalyst used in Step (i) vapor phase hydrofluorination reaction may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, such as from about 10 m²/g to about 300 m²/g, from about 20 m²/g to about 250 m²/g, from about 30 m²/g to about 225 m²/g, from about 40 m²/g to about 200 m²/g, from about 50 m²/g to about 175 m²/g, from about 60 m²/g to about 150 m²/g, from about 70 m²/g to about 140 m²/g, from about 80 m²/g to about 130 m²/g, from about 90 m²/g, to about 120 m²/g, or from about 100 m²/g to about 110 m²/g.

### Step (i) Catalyst Pretreatment

Prior to reaction, the liquid phase catalyst may be fluorinated to become an active form (e.g., a mixed metal chloride and fluoride species). Active pentahalogenated antimony catalysts include compounds of the formula SbCl₅-xFₓ (where x=1, 2, 3, 4 or 5). SbCl₃ or SbCl₅ are typically employed as catalyst precursors, which are the starting source of the active halogenated antimony catalyst. SbCl₅ can be made to become an active pentahalogenated antimony catalyst by the addition of HF. For example, an antimony pentahalogenated hydrofluorination catalyst is prepared by reacting antimony chloride, such as antimony pentachloride, with excess HF in situ. Antimony pentachloride may be charged with HF in excess prior to the addition of reactant to the reactor. Without limitation, SbCl₃ can be made to become an active pentahalogenated antimony species by the addition of Cl₂ which oxidizes it to SbCl₅ followed by fluorination by the addition of hydrogen fluoride (HF). SbCl₃ can also be made to become an active pentahalogenated antimony species by the addition of F₂ which oxidizes it to SbCl₃—F₂. To convert Sb⁺³ compounds (e.g., SbCl₃) to Sb⁺⁵ compounds (e.g., SbCl₅-xFₓ (where x=1, 2, 3, 4, or 5)), addition of Cl₂ followed by HF is preferred.

Prior to the reaction, for the vapor phase hydrofluorination reaction of Step (i), the catalyst may be pretreated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints, such as from about 200° C. to about 400° C., from about 200° C. to about 350° C., from about 250° C. to about 350° C., from about 250° C. to about 300° C., or from about 260° C. to about 300° C.

### Step (i) Reaction Temperature

For the liquid phase hydrofluorination reaction of Step (i), the reaction temperature may be as low as about 80° C., about 90° C., about 100° C., about 110° C., about 120° C., about 130° C., about 140° C., about 150° C., about 160° C., about 170° C., about 180° C., about 190° C., about 200° C., or as high as about 250° C., about 300° C., about 350° C., about 400° C., or within any range encompassed by two of the foregoing values as endpoints, such as from about 80° C. to about 400° C., for example. The temperature may preferably be from about 200° C. to about 360° C., and more preferably from about 200° C. to about 350° C. Specific examples of additional suitable ranges are set forth below in Table 1. The numerical ranges set forth in Table 1 below are understood to be prefaced by “about”.

TABLE 1
Temperature Ranges - Step (i) Hydrofluorination

| From (° C.) | To (° C.) |
|---|---|
| 80 | 400 |
| 80 | 350 |
| 80 | 300 |
| 80 | 250 |
| 80 | 200 |
| 80 | 150 |
| 100 | 400 |
| 100 | 350 |
| 100 | 300 |
| 150 | 400 |
| 150 | 350 |
| 150 | 300 |
| 200 | 400 |
| 200 | 360 |
| 200 | 350 |

### Step (i) Reaction Contact Time

For the liquid phase hydrofluorination reaction of Step (i), the contact time of reactants with the catalyst may be as little as about 0.5 hour, about 1 hours, about 2 hours, about 3 hours, about 4 hours, about 5 hours, or as long as about 6 hours, about 7 hours, about 8 hours, about 9 hours, about 10 hours, about 11 hours, about 12 hours, about 13 hours, about 14 hours, about 15 hours, or within any range encompassed by two of the foregoing values as endpoints, such as from about 0.5 hour to about 15 hours, or from about 1 hour to about 15 hours. The contact time may preferably be from about 2 hour to about 14 hours, and more preferably from about 5 hours to about 12 hours.

For the vapor phase hydrofluorination reaction of Step (i), the contact time of reactants with the catalyst may be as little as about 0.1 second, about 1 second, about 5 seconds, about 10 seconds, about 20 seconds, about 30 seconds, or as long as about 45 seconds, about 1 minute, about 1 minute 15 seconds, about 1 minute 30 seconds, about 2 minutes, or within any range encompassed by two of the foregoing values as endpoints, such as from about 0.1 second to about 2 minutes.

### Step (i) Reaction Pressure

For the hydrofluorination reaction of Step (i), the pressure inside which reactor the hydrofluorination reaction of Step (i) takes place may be as little as about 1 psig, about 10 psig, about 20 psig, about 30 psig, about 40 psig, about 50 psig, about 60 psig, about 70 psig, about 80 psig, about 90 psig, about 100 psig, or as great as about 150 psig, about 200 psig, about 250 psig, about 300 psig, about 350 psig, about 400 psig, about 450 psig, about 500 psig, or within any range encompassed by two of the foregoing values as endpoints, such as from about 1 psig to about 500 psig. The pressure may preferably be from about 50 psig to about 500 psig, and more preferably from about 70 psig to about 400 psig. Specific examples of additional suitable ranges are set forth below in Table 2. The numerical ranges set forth in Table 2 below are understood to be prefaced by “about”.

TABLE 2
Pressure Ranges - Step (i) Hydrofluorination

| From (psig) | To (psig) |
|---|---|
| 1 | 500 |
| 1 | 400 |
| 10 | 500 |
| 10 | 400 |
| 10 | 300 |
| 10 | 200 |
| 10 | 150 |
| 10 | 100 |
| 50 | 500 |
| 50 | 400 |
| 50 | 300 |
| 50 | 200 |
| 50 | 150 |
| 50 | 100 |
| 70 | 500 |
| 70 | 400 |
| 70 | 300 |
| 70 | 200 |
| 70 | 150 |
| 70 | 100 |
| 100 | 500 |
| 100 | 400 |
| 100 | 300 |
| 100 | 200 |
| 100 | 150 |

### Step (i) Reaction Conditions

A summary of the preferred catalyst, temperatures, and pressures as discussed above are summarized in Table 3 below. The numerical ranges set forth in Table 3 are understood to be prefaced by “about”.

TABLE 3
Summary of Preferred Step (i) Hydrofluorination Conditions

| Catalyst | Temperature (° C.) | Pressure (psig) |
|---|---|---|
| SbCl₅ | 80-400 | 1-400 |
| SbCl₅ | 80-400 | 50-400 |
| SbCl₅ | 80-400 | 100-200 |
| SbCl₅ | 80-400 | 100-150 |
| SbCl₅ | 80-200 | 1-400 |
| SbCl₅ | 80-200 | 50-400 |
| SbCl₅ | 80-200 | 100-200 |
| SbCl₅ | 80-200 | 100-150 |
| SbCl₅ | 80-100 | 1-400 |
| SbCl₅ | 80-100 | 50-400 |
| SbCl₅ | 80-100 | 100-200 |
| SbCl₅ | 80-100 | 100-150 |
| TaCl₅ | 80-400 | 1-400 |
| TaCl₅ | 80-400 | 50-400 |
| TaCl₅ | 80-400 | 100-200 |
| TaCl₅ | 80-400 | 100-150 |
| TaCl₅ | 80-200 | 1-400 |
| TaCl₅ | 80-200 | 50-400 |
| TaCl₅ | 80-200 | 100-200 |
| TaCl₅ | 80-200 | 100-150 |
| TaCl₅ | 80-100 | 1-400 |
| TaCl₅ | 80-100 | 50-400 |
| TaCl₅ | 80-100 | 100-200 |
| TaCl₅ | 80-100 | 100-150 |
| SnCl₄ | 80-400 | 1-400 |
| SnCl₄ | 80-400 | 50-400 |
| SnCl₄ | 80-400 | 100-200 |
| SnCl₄ | 80-400 | 100-150 |
| SnCl₄ | 80-200 | 1-400 |
| SnCl₄ | 80-200 | 50-400 |
| SnCl₄ | 80-200 | 100-200 |
| SnCl₄ | 80-200 | 100-150 |
| SnCI₄ | 80-100 | 1-400 |
| SnCl₄ | 80-100 | 50-400 |
| SnCl₄ | 80-100 | 100-200 |
| SnCl₄ | 80-100 | 100-150 |
| SbF₃ | 80-400 | 1-400 |
| SbF₃ | 80-400 | 50-400 |
| SbF₃ | 80-400 | 100-200 |
| SbF₃ | 80-400 | 100-150 |
| SbF₃ | 80-200 | 1-400 |
| SbF₃ | 80-200 | 50-400 |
| SbF₃ | 80-200 | 100-200 |
| SbF₃ | 80-200 | 100-150 |
| SbF₃ | 80-100 | 1-400 |
| SbF₃ | 80-100 | 50-400 |
| SbF₃ | 80-100 | 100-200 |
| SbF₃ | 80-100 | 100-150 |
| Cr₂O₃ | 80-400 | 1-400 |
| Cr₂O₃ | 80-400 | 50-400 |
| Cr₂O₃ | 80-400 | 50-200 |
| Cr₂O₃ | 80-400 | 50-100 |
| Cr₂O₃ | 100-300 | 1-400 |
| Cr₂O₃ | 100-300 | 50-400 |
| Cr₂O₃ | 100-300 | 50-200 |
| Cr₂O₃ | 100-300 | 50-100 |
| Cr₂O₃ | 200-300 | 1-400 |
| Cr₂O₃ | 200-300 | 50-400 |
| Cr₂O₃ | 200-300 | 50-200 |
| Cr₂O₃ | 200-300 | 50-100 |

### Step (i) Reactant Mole Ratio

For the hydrofluorination reaction of Step (i), the mole ratio of hydrogen fluoride (HF) to HCC-260fb may be as little about 1:1, about 2:1, about 3:1, about 4:1, about 5:1, about 6:1, about 7:1, about 8:1, about 9:1, as great as about 10:1, about 15:1, about 20:1 or about 25:1, about 30:1, about 40:1, about 50:1, or within any range encompassed by two of the foregoing values as endpoints, for example, from about 2:1 to about 40:1. The mole ratio of HF to HCC-260 may be preferably from about 2:1 about to 30:1, and more preferably from about 2:1 to about 20:1.

### Step (i) Conversion of the Starting Material

As demonstrated by the Examples herein, the hydrofluorination reaction of Step (i) may achieve a conversion of the starting material of greater than about 30%, greater than about 35%, greater than about 40%, greater than about 45%, greater than about 50%, and for each of the foregoing, less than or equal to 100%, or within any range encompassed by two of the foregoing values as endpoints, such as from about 30% to about 100%, from about 35% to about 100%, or from about 40% to about 100%.

### Step (i) Selectivity to HFC-263fb

As demonstrated by the Examples herein, the hydrofluorination of Step (i) may achieve a selectivity to the desired product HCFC-262fc of at least about 50%, at least about 60%, at least about 70%, at least about 80%, at least about 90%, and for each of the foregoing, less than or equal to 100%, or within any range encompassed by two of the foregoing values as endpoints, such as from about 50% to about 100%, or about 60% to about 100%, based on total moles of the organic components of the composition.

### Step (i) Product Composition

As discussed above, the hydrofluorination reaction of Step (i) produces a product mixture comprising 1,1-dichloro-1-fluoropropane (HCFC-261fc), 1-chloro-1,1-difluoropropane (HCFC-262fc), 1,1,1-trifluoropropane (HFC-263fb), HCl, and unconverted raw materials including HF and 1,1,1-trichloropropane (HCC-260fb).

For a hydrofluorination reaction of Step (i) carried out using suitable catalysts as described herein, referring to FIG. 1, the amount of HCFC-262fc in the organic product composition in stream 106 from the reactor 104 may be at least 20 mol %, at least 30 mol %, at least 40 mol %, at least 50 mol %, at least 60 mol %, at least 70 mol %, at least 75 mol %, at least 80 mol %, at least 85 mol %, or at least 90 mol %, and for each of the foregoing, less than or equal to 100 mol %, for example, based on total moles of organic components of the composition.

For a hydrofluorination reaction of Step (i) carried out using suitable catalysts as described herein, referring to FIG. 1, the amount of unconverted HCC-260fb, if present, may be greater than or equal to 0.1 mol % and yet less than 70 mol %, less than 60 mol %, less than 50 mol %, less than 40 mol %, less than 30 mol %, less than 20 mol %, less than 10 mol %, less than 9 mol %, less than 8 mol %, less than 7 mol %, less than 6 mol %, or less than 5 mol %, for example, based on total moles of organic components of the composition.

For a hydrofluorination reaction of Step (i) carried out using suitable catalysts as described herein, referring to FIG. 1, the amount of HCFC-261fc, if present, may be greater than or equal to 0.1 mol % and yet less than 10 mol %, less than 9 mol %, less than 8 mol %, less than 7 mol %, less than 6 mol %, or less than 5 mol %, for example, based on total moles of organic components of the composition.

For a hydrofluorination reaction of Step (i) carried out using suitable catalysts as described herein, referring to FIG. 1, the amount of HFC-263fb, if present, may be greater than or equal to 0.1 mol % and yet less than 10 mol %, less than 9 mol %, less than 8 mol %, less than 7 mol %, less than 6 mol %, or less than 5 mol %, for example, based on total moles of organic components of the composition.

For a hydrofluorination reaction of Step (i) carried out using suitable catalysts as described herein, referring to FIG. 1, the amount of other byproducts, if present, may be greater than or equal to 0.1 mol % and yet less than 10 mol %, less than 9 mol %, less than 8 mol %, less than 7 mol %, less than 6 mol %, or less than 5 mol %, for example, based on total moles of organic components of the composition.

For example, for a hydrofluorination reaction of Step (i) carried out using suitable catalysts as described herein, referring to FIG. 1, in the product composition in stream 106 from the reactor 104, the amount of HCFC-262fc may be at least 80 mol % and less than or equal to 99.8 mol %, the amount of unconverted HCC-260fb may be at least 0.1 mol % and less than or equal to 10 mol %, and the amount of HCFC-261fc may be at least 0.1 mol % and less than or equal to 10 mol %, based on the combined total moles of the HCFC-262fc, HCC-260fb, and HCFC-261fc in the product composition.

The combined amount of HCFC-262fc, HCC-260fb, and HCFC-261fc in the composition set forth in the preceding paragraph may be at least 80 mol % and less than or equal to 100% of the total moles of organic components of the product composition in stream 106 from the reactor 104, while the combined amount of other components including undesired byproducts (e.g., HFC-263fb) may be greater than or equal to 0 mol % and less than 20 mol % of the total moles of organic components of the product composition in stream 106 from the reactor 104.

For example, for a hydrofluorination reaction of Step (i) carried out using suitable catalysts as described herein, referring to FIG. 1, in the product composition in stream 106 from the reactor 104, the amount of HCFC-262fc may be at least 85 mol % and less than or equal to 99.8 mol %, the amount of unconverted HCC-260fb may be at least 0.1 mol % and less than or equal to 7.5 mol %, and the amount of HCFC-261fc may be at least 0.1 mol % and less than or equal to 7.5 mol %, based on the combined total moles of the HCFC-262fc, HCC-260fb, and HCFC-261fc in the product composition.

The combined amount of HCFC-262fc, HCC-260fb, and HCFC-261fc in the composition set forth in the preceding paragraph may be at least 85 mol % and less than or equal to 100% of the total moles of organic components of the product composition in stream 106 from the reactor 104, while the combined amount of other components including undesired byproducts (e.g., HFC-263fb) may be greater than or equal to 0 mol % and less than 15 mol % of the total moles of organic components of the product composition in stream 106 from the reactor 104.

For example, for a hydrofluorination reaction of Step (i) carried out using suitable catalysts as described herein, referring to FIG. 1, in the product composition in stream 106 from the reactor 104, the amount of HCFC-262fc may be at least 90 mol % and less than or equal to 99.8 mol %, the amount of unconverted HCC-260fb may be at least 0.1 mol % and less than or equal to 5 mol %, and the amount of HCFC-261fc may be at least 0.1 mol % and less than or equal to 5 mol %, based on the combined total moles of the HCFC-262fc, HCC-260fb, and HCFC-261fc in the product composition.

The combined amount of HCFC-262fc, HCC-260fb, and HCFC-261fc in the composition set forth in the preceding paragraph may be at least 90 mol % and less than or equal to 100% of the total moles of organic components of the product composition in stream 106 from the reactor 104, while the combined amount of other components including undesired byproducts (e.g., HFC-263fb) may be greater than or equal to 0 mol % and less than 10 mol % of the total moles of organic components of the product composition in stream 106 from the reactor 104.

### V. Step (ii)-(a) Vapor Phase

The dehydrochlorination of 1-chloro-1,1-difluoropropane (HCFC-262fc) can be carried out in a vapor phase reactor in the absence of any catalyst. The said vapor phase reactor may be a bundle-type tubular reactor or an electrical heater reactor as described under the Section III—Process Flow. Step (ii)-(a) of Process 1 includes dehydrochlorinating HCFC-262fc in vapor phase to produce HFO-1252zc. The process conditions used in Step (ii)-(a) vapor phase dehydrochlorination reaction of Process 1 is detailed below.

Step (ii)-(a) Reaction Temperature

The temperature range for the dehydrochlorination reaction of Step (ii)-(a) may be as low as about 150° C., about 200° C., about 250° C., about 300° C., about 350° C., about 400° C., about 450° C., or as high as about 500° C., about 550° C., about 600° C., about 650° 1, about 700° C., about 750° C., about 800° C. or within any range encompassed by two of the foregoing values as endpoints, such as from about 150° C. to about 800° C., or from about 200° C. to about 700° C. The temperature may be preferably from about 300° C. to about 600° C., and more preferably from about 400° C. to about 500° C. Specific examples of additional suitable ranges are set forth below in Table 4. The numerical ranges set forth in Table 4 below are understood to be prefaced by “about”.

TABLE 4
Temperature Ranges - Step (ii)-a Vapor Phase Dehydrochlorination

| From (° C.) | To (° C.) |
|---|---|
| 150 | 800 |
| 150 | 700 |
| 150 | 600 |
| 150 | 500 |
| 200 | 800 |
| 200 | 700 |
| 200 | 600 |
| 200 | 500 |
| 300 | 800 |
| 300 | 700 |
| 300 | 600 |
| 300 | 500 |
| 400 | 800 |
| 400 | 700 |
| 400 | 600 |
| 400 | 500 |

Step (ii)-(a) Reaction Pressure

The pressure within the reactor where the dehydrochlorination reaction of Step (ii)-(a) takes place may be as little as about 5 psig, about 10 psig, about 15 psig, about 20 psig, about 25 psig, about 30 psig, about 35 psig, about 40 psig, about 45 psig, about 50 psig, about 55 psig, about 60 psig, about 65 psig, about 70 psig, or as high as about 100 psig, as about 150 psig, as about 200 psig, as about 250 psig, as about 300 psig, as about 350 psig, as about 400 psig, as about 450 psig, as about 500 psig, or within any range encompassed by two of the foregoing values as endpoints, such as from about 5 psig to about 500 psig. For example, the pressure may preferably be from about 50 psig to about 250 psig, and more preferably from about 70 psig to about 150 psig. Specific examples of additional suitable ranges are set forth below in Table 5. The numerical ranges set forth in Table 5 below are understood to be prefaced by “about”.

TABLE 5
Pressure Ranges - Step (ii)-a Vapor Phase Dehydrochlorination

| From (psig) | To (psig) |
|---|---|
| 5 | 500 |
| 20 | 500 |
| 20 | 400 |
| 20 | 300 |
| 20 | 200 |
| 50 | 500 |
| 50 | 400 |
| 50 | 350 |
| 50 | 300 |
| 50 | 250 |
| 50 | 200 |
| 50 | 150 |
| 70 | 500 |
| 70 | 400 |
| 70 | 350 |
| 70 | 300 |
| 70 | 250 |
| 70 | 200 |
| 70 | 150 |

Step (ii)-a Reaction Conditions

A summary of the preferred temperatures and pressures as discussed above are summarized in Table 6 below. The numerical ranges set forth in Table 6 are understood to be prefaced by “about”.

TABLE 6
Summary of Preferred Step (ii)-a Vapor Phase Dehydrochlorination

| Temperature (° C.) | Pressure (psig) |
|---|---|
| 150-800 | 5-500 |
| 150-800 | 10-400 |
| 150-800 | 50-300 |
| 150-800 | 50-250 |
| 150-800 | 50-200 |
| 150-800 | 50-150 |
| 150-800 | 50-100 |
| 150-800 | 70-300 |
| 150-800 | 70-250 |
| 150-800 | 70-200 |
| 150-800 | 70-150 |
| 150-800 | 70-100 |
| 200-600 | 5-500 |
| 200-600 | 10-400 |
| 200-600 | 50-300 |
| 200-600 | 50-250 |
| 200-600 | 50-200 |
| 200-600 | 50-150 |
| 200-600 | 50-100 |
| 200-600 | 70-300 |
| 200-600 | 70-250 |
| 200-600 | 70-200 |
| 200-600 | 70-150 |
| 200-600 | 70-100 |
| 300-500 | 5-500 |
| 300-500 | 10-400 |
| 300-500 | 50-300 |
| 300-500 | 50-250 |
| 300-500 | 50-200 |
| 300-500 | 50-150 |
| 300-500 | 50-100 |
| 300-500 | 70-300 |
| 300-500 | 70-250 |
| 300-500 | 70-200 |
| 300-500 | 70-150 |
| 300-500 | 70-100 |
| 400-500 | 5-500 |
| 400-500 | 10-400 |
| 400-500 | 50-300 |
| 400-500 | 50-250 |
| 400-500 | 50-200 |
| 400-500 | 50-150 |
| 400-500 | 50-100 |
| 400-500 | 70-300 |
| 400-500 | 70-250 |
| 400-500 | 70-200 |
| 400-500 | 70-150 |
| 400-500 | 70-100 |

Step (ii)-(a) Conversion of the Starting Material

As demonstrated by the Examples herein, the dehydrochlorination reaction of Step (ii)-(a) may achieve a conversion of the starting material of greater than about 20%, greater than about 25%, greater than about 30%, greater than about 35%, greater than about 40%, greater than about 45%, greater than about 50%, and for each of the foregoing, less than or equal to 100%, or within any range encompassed by two of the foregoing values as endpoints, such as from about 30% to about 100%, from about 35% to about 100%, or from about 40% to about 100%.

Step (ii)-(a) Selectivity to HFO-1252zc

As demonstrated by the Examples herein, the selectivity to the desired product HFO-1252zc for the dehydrochlorination reaction of Step (ii)-(a) may be greater than about 80%, about 85%, about 90%, about 91%, about 92%, about 93%, about 94%, about 95% about 96%, about 97%, about 98%, about 99%, and for each of the foregoing, less than or equal to 100%, or within any range encompassed by two of the foregoing values as endpoints, such as from about 90% to about 100%, or from about 95% to about 100%.

### VI. Step (ii)-(b)

As an alternative to a vapor phase reaction, the dehydrochlorination reaction of Step (ii) may also be carried out in a suitable liquid phase reactor charged with a basic solution. Step (ii)-(b) of Process 1 includes dehydrochlorinating 1-chloro-1,1-difluoropropane (HCFC-262fc) in liquid phase to produce HFO-1252zc. The liquid phase reactants and process conditions used in Step (ii)-(b) liquid phase dehydrochlorination reaction of Process 1 is detailed below.

Step (ii)-(b) Caustic Solution

The dehydrochlorination reaction of Step (ii)-(b) may be carried out in a liquid phase reactor 114 in the presence of a caustic solution. Suitable bases that may be used to make the caustic solution include, but are not limited to, potassium hydroxide (KOH), sodium hydroxide (NaOH), calcium oxide (CaO), lithium oxide (LiOH), and alkaline earth metal hydroxides such as calcium hydroxide (Ca(OH)₂), magnesium hydroxide (Mg(OH)₂), strontium hydroxide (Sr(OH)₂), and mixtures thereof.

The caustic solution containing feed stream in reactor 114 can be an aqueous mixture, and can contain the base at any suitable strength for producing the desired reaction. For example, when KOH is used as the base, the base containing feed stream can contain base at a strength from about 5 wt. % to about 55 wt. %, preferably from about 25 wt. % to about 50 wt. %.

Step (ii)-(b) Optional Catalyst

The dehydrochlorination reaction of Step (ii)-(b) may be carried out in a liquid phase reactor 114 optionally in the presence of a phase transfer agent or catalyst for enhancing the reaction rate. Suitable phase transfer catalyst include, but are not limited to, behentrimonium chloride (C₂₅H₅₄ClN) (e.g., Aliquat®336). Other phase transfer catalysts that can be used include, but not limited to, quaternary ammonium salts, quaternary phosphonium salts, quaternary arsenium salts, sulfonium salts, crown ethers and similar molecules. In some examples, the optional catalyst is preferably a quaternary ammonium salt or a quaternary phosphonium salt.

Step (ii)-(b) Reaction Temperature

For a liquid phase dehydrochlorination reaction of Step (ii)-(b) of Process 1 using suitable bases as described herein, the reaction temperature of Step (ii)-(b) may be as low as about 30° C., about 35° C., about 40° C., about 45° C., about 50° C., or as high as about 100° C., about 110° C., about 120° C., about 130° C., about 140° C., about 150° C., or within any range encompassed by two of the foregoing values as endpoints, such as from about 30° C. to about 150° C., preferably from about 40° C. to about 130° C., or more preferably from about 50° C. to about 120° C., for example. Specific examples of additional suitable ranges are set forth below in Table 7. The numerical ranges set forth in Table 7 below are understood to be prefaced by “about”.

TABLE 7
Temperature Ranges - Step (ii)-b
Liquid Phase Dehydrochlorination

| From (° C.) | To (° C.) |
|---|---|
| 30 | 150 |
| 30 | 130 |
| 30 | 120 |
| 30 | 100 |
| 40 | 150 |
| 40 | 130 |
| 40 | 120 |
| 40 | 100 |
| 50 | 150 |
| 50 | 130 |
| 50 | 120 |
| 50 | 100 |

Step (ii)-(b) Conversion of the Starting Material

As demonstrated by the Examples herein, the dehydrochlorination reaction of Step (ii)-(b) may achieve a conversion of the starting material of greater than about 20%, greater than about 25%, greater than about 30%, greater than about 35%, greater than about 40%, greater than about 45%, greater than about 50%, and for each of the foregoing, less than or equal to 100%, or within any range encompassed by two of the foregoing values as endpoints, such as from about 30% to about 100%, from about 35% to about 100%, or from about 40% to about 100%.

Step (ii)-(b) Selectivity to HFO-1252zc

As demonstrated by the Examples herein, the selectivity to the desired product HFO-1252zc for the dehydrochlorination reaction of Step (ii)-(a) may be greater than about 80%, about 85%, about 90%, about 91%, about 92%, about 93%, about 94%, about 95% about 96%, about 97%, about 98%, about 99%, and for each of the foregoing, less than or equal to 100%, or within any range encompassed by two of the foregoing values as endpoints, such as from about 90% to about 100%, or from about 95% to about 100%.

### EXAMPLES

### Example 1a

### Hydrofluorination of 1,1,1-Trichloropropane (HCC-260fb) in a Liquid Phase Reactor

The hydrofluorination of 1,1,1-trichloropropane (HCC-260fb) is performed in a 1-gallon Hastelloy C276 Parr reactor, equipped with agitator, heater, temperature indicator and pressure transducer. Vapor from reactor is sent to catalyst stripper/recycle column (1″ OD×18″ long) having about a 10 stages. The vapor from top of the distillation column is sent to the top of a condenser. Coolant is fed to the bottom of the condenser. Therefore, this is countercurrent heat exchanger. The vapor product is sent to a 10 wt % KOH caustic solution scrubber for acid removal followed by a drying column for moisture removal before being condensed and collected into a Product Collection Cylinder (PCC). The liquid is fed back into the distillation column and then back into reactor.

1000 g of SbCl₅ catalyst is fed into the Parr reactor. Then the Parr reactor is heated to the reaction temperature of 90° C. 200 g of AHF is fed into the Parr reactor to partially fluorinate SbCl₅ into SbCl₅-xFₓ (0<x<4). During the fluorination of SbCl₅, HCl is formed and is released into a 10 wt % KOH caustic solution carboy for neutralization. Upon the completion of catalyst fluorination, the Parr reactor is cooled to below 30° C. 2200 g of HCC-260fb and 900 g of AHF (AHF/HCC-260fb mole ratio=3/1) are fed to the reactor and the whole is heated and held at 90° C. The reactor pressure is maintained between 100 psig and 150 psig during the reaction.

After 10 hours, the reaction is deemed to be completed. All the reactor contents are slowly vented off and all organic contents are collected into PCC. Heat is applied during vent-off process to drive off all reactor contents. 1565 g of organic is recovered and analyzed by means of GC-MS and GC. The results show the organic contents contain <5% HCC-260fb, <5% HCFC-261fc, about 75% HCFC-262fc, and <5% HFC-263fb, and <5% others.

### Example 1b

### Hydrofluorination of 1,1,1-Trichloropropane (HCC-260fb) in a Vapor Phase Reactor

This example illustrates the continuous vapor phase fluorination reaction of 1,1,1-trichloropropane (HCC-260fb) to 1-chloro-1,1-difluoropropane (HCFC-262fc). The fluorination catalyst for the experiment is fluorinated Cr₂O₃.

A continuous vapor phase fluorination reaction system consisting of N₂, HF, and organic feed systems, feed vaporizer, superheater, 2 inch ID Monel reactor, acid scrubber, drier, and product collection system is used to study the reaction. The reactor is about 33 inches in length. The reactor is loaded with 1.8 liters of pretreated fluorinated Cr₂O₃ catalyst. The reactor is then heated to a temperature of about 180° C. with a N₂ purge going over the catalyst after the reactor has been installed in a constant temperature sand bath. HF feed at a feed rate of 1.0 lb/h is introduced to the reactor (via the vaporizer and superheater) as a co-feed with the N₂ for 15 minutes when the N₂ flow is stopped. The HF flow rate is adjusted to 2.2 lb/hr and then 1,1,1-trichloropropane (HCC-260fb) feed is started to the reactor (via the vaporizer and superheater). The feed rate of HCC-260fb is kept steady at 1.0 lb/hr and HF feed is kept steady at 2.2 lb/hr for about 16 to 1 mole ratio of HF to HCC-260fb.

Once the reaction starts the catalyst bed temperature rises to a temperature ranged from about 200° C. to about 220° C. The reaction temperature is gradually increased as catalyst deactivation occurs to maintain desired product collection rate, and reaction is stopped once the reaction temperature reached 300° C. and the conversion of HCC-260fb is <30%. The reaction pressure is kept constant at 70 psig during the entire course of reaction. The reaction is continuously run for about 600 hours before the experiment ends. 255 lb of HCFC-262fc crude material is produced. The average conversion of HCC-260fb and the average selectivity to HCFC-262fc are 71%, and 86%, respectively.

The catalyst is then regenerated using oxygen and high temperature to burn off the coke that is coating the surface of the catalyst. The reaction is restarted as before and the catalyst has regained its activity. The initial HCC-260fb conversion is >97% and the initial HCFC-262fc selectivity is >95% at a catalyst bed hot spot temperature of 200-210° C. and at a pressure of 70 psig.

### Example 2

### Dehydrochlorination of 1-Chloro-1,1-Difluoropropane (HCFC-262fc) in an Inconel 625 Tubular Reactor

A cylindrical Inconel 625 reactor of ¾″ diameter immersed into a 3-zone electrical furnace is used. Process temperatures are recorded using a multi-point thermocouple placed inside the reactor. The hottest middle 3 points are controlled to be close to each other and are considered as the reaction zone with a volume of about 20 ml. The HCFC-262fc feed material is of 99.9 mol % purity. HCFC-262fc is fed into the bottom of the vertically mounted reactor and is vaporized before reaching reaction zone. The flow rate of HCFC-262fc is 12 g/h in a typical run. Effluent gases are passed through a gas sampling tube and the progress of the reaction is monitored periodically via GC analysis of the contents of the gas sampling tube. HCFC-262fc conversions at 450° C. and 70 psig are generally between 30 and 40% and no deactivation is noted during the period of time of the test which lasts for about 100 hours. The selectivity to HFO-1252zc remains high at around 97% during the reaction.

### Example 3

### Dehydrochlorination of 1-Chloro-1,1-Difluoropropane (HCFC-262fc) in an Inconel 600 Electric Heater Reactor

The electric heater reactor includes the outer Inconel 600 tube and the inside heating element. The heating element has a thermal resistance wire, MgO ceramic shell, and an Inconel 600 sheath. The heating element is placed inside a % inch Inconel 600 tube and there are four thermocouples inserted into the Inconel tube from the side so that their tip is touching the inside heating element's surface (providing the skin temperature). The reactive zone has a volume of about 45 ml. The HCFC-262fc feed material is of 99.9 mol % purity. HCFC-262fc is fed into the bottom of the electric heater reactor at a flow rate of 30 g/h in a typical run. Effluent gases are passed through a gas sampling tube and the progress of the reaction is monitored periodically via GC analysis of the contents of the gas sampling tube. HCFC-262fc conversions at 450° C. and 70 psig are generally between 30 and 40% and no deactivation is noted during the period of time of the test which lasts for about 100 hours. The selectivity to HFO-1252zc remains high at around 97% during the reaction.

### Example 4

### Dehydrochlorination of 1-Chloro-1,1-Difluoropropane (HCFC-262fc) Using KOH Caustic Solution

A 1-liter Parr reactor equipped with agitator is used to convert HCFC-262fc to HFO-1252zc. 15 g of Aliquat 336 is added to reactor. 224 g of 99.9% HCFC-262fc and 370 g of 45% KOH are added to reactor. Reactor temperature of reaction is slowly increased to 60 C. The reaction is run for 6 hours at 60 C. After reaction, the organic content is vented off to a chilled product collection cylinder. GC analysis shows the organic content contains 65 mol % of HFO-1252zc and 33% of HCFC-262fc.

### Examples 5-7

### Hydrofluorination of 1,1,1-Trichloropropane (HCC-260fb) Using Various Catalysts

The hydrofluorination of HCC-260fb is performed using a process similar to Example 1 with different catalysts. Similar results are achieved with HCFC-262fc produced as a main product in the product composition.

TABLE 1
Catalysts and Reaction Conditions for Examples 5-7

| Example | Catalyst | Temperature | Pressure | Contact Time |
|---|---|---|---|---|
| 5 | TaCl₅ | 90° C. | 100-150 psig | 10 hours |
| 6 | SnCl₄ | 90° C. | 100-150 psig | 10 hours |
| 7 | SbF₃ | 90° C. | 100-150 psig | 10 hours |

### ASPECTS

Aspect 1 is a method for producing 1,1-difluoropropene (HFO-1252zc), comprising: reacting 1,1,1-trichloropropane (HCC-260fb) with hydrogen fluoride in the presence of a catalyst to produce a first product composition comprising 1-chloro-1,1-difluoropropane (HCFC-262fc); and reacting HCFC-262fc from the first product composition to produce a second product composition comprising 1,1-difluoropropene (HFO-1252zc).

Aspect 2 is the method of Aspect 1, wherein the first reacting step is conducted in a liquid phase at a temperature of from about 50° C. to about 250° C.

Aspect 3 is the method of Aspects 1 or 2, wherein the catalyst is an antimony halide or a tantalum halide.

Aspect 4 is the method of any one of Aspects 1 to 3, wherein the catalyst is selected from at least one of SbCl₅, TaCl₅, SnCl₄, SbF₃, and combinations of the foregoing.

Aspect 5 is the method of any one of Aspects 1 to 4, wherein the catalyst is fluorinated SbCl₅.

Aspect 6 is the method of Aspect 1, wherein the first reacting step is conducted in a vapor phase at a temperature of from about 200° C. to about 400° C.

Aspect 7 is the method of Aspect 6, wherein the catalyst is an fluorinated chromia and fluorinated zinc oxide-chromia.

Aspect 8 is the method of Aspects 6 or 7, wherein the catalyst is selected from at least one of fluorinated metal oxides including Cr₂O₃, Al₂O₃, ZnO—Cr₂O₃, and combinations of the foregoing.

Aspect 9 is the method of any one of Aspects 6 to 8, wherein the catalyst is selected from at least one of metal halides including CrF₃, AlF₃, and combinations of the foregoing.

Aspect 10 is the method of any one of Aspects 1 to 9, wherein the mole ratio of hydrogen fluoride to HCC-260fb is from about 2:1 to about 50:1.

Aspect 11 is the method of any one of Aspects 1 to 10, wherein the first product composition comprises: 80 mol % to 99.8 mol % HCFC-262fc; 0.1 mol % to 10 mol % 1,1-dichloro-1-fluoropropane (HCFC-261fc), and 0.1 mol % to 10 mol % HCC-260fb, based on the combined total moles of the HCFC-262fc, HCC-260fb, and HCFC-261fc in the product composition.

Aspect 12 is the method of Aspect 11, wherein the first product composition comprises a total amount of HCFC-262fc, HCC-260fb, and HCFC-261fc of at least 80 mol %, based on total moles of organic components of the first product composition.

Aspect 13 is the method of any one of Aspects 1 to 12, wherein the second reacting step is conducted in vapor phase.

Aspect 14 is the method of Aspect 13, wherein the second reacting step is conducted without the presence of a catalyst in an electric heater reactor.

Aspect 15 is the method of Aspects 13 or 14, wherein the second step is conducted from about 150° C. to about 800° C.

Aspect 16 is the method of any one of Aspects 13 to 15, wherein the second product composition comprises: 25 mol % to 99.9 mol % 1,1-difluoropropene (HFO-1252zc); and 0.1 mol % to 75 mol % 1,1,1-trifluoropropane (HFC-263fb), based on the combined total moles of the HFC-263fb and HFO-1252zc in the second product composition.

Aspect 17 is the method of Aspect 16, wherein the second reacting step achieves a conversion of HCFC-262fc of from about 30% to about 100%.

Aspect 18 is the method of any one of Aspects 13 to 17, wherein the second reacting step achieves a selectivity to HFO-1252zc of from about 90% to about 100%.

Aspect 19 is the method of any one of Aspects 1 to 12, wherein the second reacting step comprises reacting HCFC-262fc from the first product composition in the presence of a caustic solution to produce the second product composition.

Aspect 20 is the method of Aspect 19, wherein the caustic solution comprises a base selected from at least one of KOH, NaOH, CaO, LiOH, Ca(OH)₂, Mg(OH)₂, Sr(OH)₂, and combinations of the foregoing.

Aspect 21 is the method of Aspects 19 or 20, wherein the second reacting step is conducted at a second temperature of from about 30° C. to about 150° C.

Aspect 22 is the method of any one of Aspects 19 to 21, wherein the second reacting step is conducted in the presence of a phase transfer catalyst.

Aspect 23 is the method of Aspect 22, wherein the phase transfer catalyst comprises a salt such as (but not limited to) a quaternary ammonium salt (e.g., behentrimonium chloride) or a quaternary phosphonium salt (e.g., hexadecyltributylphosphonium bromide).

Aspect 24 is the method of any one of Aspects 19 to 23, further comprising: distilling the second product composition to produce an end product composition comprising at least 99 mol % HFO-1252zc.

Aspect 25 is a product composition comprising HFO-1252zc made according to the method of any one of Aspects 1 to 24.

What is claimed is:

1. A method for producing 1,1-difluoropropene (HFO-1252zc), comprising:
reacting 1,1,1-trichloropropane (HCC-260fb) with hydrogen fluoride in the presence of a catalyst to produce a first product composition comprising 1-chloro-1,1-difluoropropane (HCFC-262fc); and
reacting HCFC-262fc from the first product composition to produce a second product composition comprising 1,1-difluoropropene (HFO-1252zc).

2. The method of claim 1, wherein the first reacting step is conducted in a liquid phase at a temperature of from about 50° C. to about 250° C.

3. The method of claim 1, wherein the catalyst is an antimony halide or a tantalum halide.

4. The method of claim 1, wherein the first reacting step is conducted in a vapor phase at a temperature of from about 200° C. to about 400° C.

5. The method of claim 4, wherein the catalyst is a fluorinated chromia and fluorinated zinc oxide-chromia.

6. The method of claim 1, wherein the mole ratio of hydrogen fluoride to HCC-260fb is from about 2:1 to about 50:1.

7. The method of claim 1, wherein the first product composition comprises:
80 mol % to 99.8 mol % HCFC-262fc;
0.1 mol % to 10 mol % 1,1-dichloro-1-fluoropropane (HCFC-261fc), and
0.1 mol % to 10 mol % HCC-260fb, based on the combined total moles of the HCFC-262fc, HCC-260fb, and HCFC-261fc in the product composition.

8. The method of claim 1, wherein the second reacting step is conducted in vapor phase.

9. The method of claim 8, wherein the second reacting step is conducted without the presence of a catalyst in an electric heater reactor.

10. The method of claim 8, wherein the second step is conducted from about 150° C. to about 800° C.

11. The method of claim 8, wherein the second product composition comprises:
25 mol % to 99.9 mol % 1,1-difluoropropene (HFO-1252zc); and
0.1 mol % to 75 mol % 1,1,1-trifluoropropane (HFC-263fb), based on the combined total moles of the HFC-263fb and HFO-1252zc in the second product composition.

12. The method of claim 11, wherein the second reacting step achieves a conversion of HCFC-262fc of from about 30% to about 100%.

13. The method of claim 8, wherein the second reacting step achieves a selectivity to HFO-1252zc of from about 90% to about 100%.

14. The method of claim 1, wherein the second reacting step comprises reacting HCFC-262fc from the first product composition in the presence of a caustic solution to produce the second product composition.

15. The method of claim 14, wherein the caustic solution comprises a base selected from at least one of KOH, NaOH, CaO, LiOH, Ca(OH)₂, Mg(OH)₂, Sr(OH)₂, and combinations of the foregoing.

16. The method of claim 14, wherein the second reacting step is conducted at a second temperature of from about 30° C. to about 150° C.

17. The method of claim 14, wherein the second reacting step is conducted in the presence of a phase transfer catalyst.

18. The method of claim 17, wherein the phase transfer catalyst comprises a salt such as (but not limited to) a quaternary ammonium salt (e.g., behentrimonium chloride) or a quaternary phosphonium salt (e.g.,hexadecyltributylphosphonium bromide).

19. The method of claim 14, further comprising:
distilling the second product composition to produce an end product composition comprising at least 99 mol % HFO-1252zc.

20. A composition comprising HFO-1252zc made according to the method of claim 1.
